(19) United States
(12) Patent Application Publication
(10) Pub. No.: US 2023/0255735 A1
(43) Pub. Date: Aug. 17, 2023

(54) CONTROLLING METHOD OF WATER FLOSSER, WATER FLOSSER CONTROL SYSTEM, AND WATER FLOSSER

(71) Applicant: Xiamen Solex High-Tech Industries Co., Ltd., Xiamen (CN)

(72) Inventors: Wei LU, Xiamen (CN); Mingfu ZHANG, Xiamen (CN); Tiancheng LI, Xiamen (CN)

(21) Appl. No.: 18/136,392

(22) Filed: Apr. 19, 2023

Related U.S. Application Data
(63) Continuation of application No. PCT/CN2021/124882, filed on Oct. 20, 2021.

(30) Foreign Application Priority Data
Oct. 20, 2020 (CN) 202011125261.8

(51) Int. Cl. A61C 17/02; G06T 7/00

(52) U.S. Cl. CPC A61C 17/0202; G06T 7/0012; G06T 2207/30036

(57) ABSTRACT

A controlling method of a water flosser, a water flosser control system, and a water flosser are provided. The controlling system comprises obtaining an oral image through an image acquiring device installed on the water flosser and controlling a water outlet nozzle of the water flosser to discharge water based on the oral image acquired by the image acquiring device.

### RELATED APPLICATIONS

This application is a continuation of and claims priority to international patent application number PCT/CN2021/124882, filed on Oct. 20, 2021, which claims priority to Chinese patent application number 202011125261.8, filed on Oct. 20, 2020. International patent application number PCT/CN2021/124882 and Chinese patent application number 202011125261.8 are incorporated herein by reference.

### FIELD OF THE DISCLOSURE

The present disclosure relates to a technical field of oral care, in particular to a controlling method, a water flosser control system, and a water flosser.

### BACKGROUND OF THE DISCLOSURE

As a personal care appliance in the field of oral health care, a water flosser uses a water pump to pump water in a water tank to a water outlet nozzle to form a jet, so as to rinse a user's mouth and teeth. Compared with ordinary toothbrushes, the water flosser is gradually accepted by consumers because of its advantages of more thorough removal of dental plaque and more scientific and effective reduction of oral diseases including gingivitis, periodontal disease, and bleeding gums, and the water flosser are gradually favored by the consumers.

When cleaning an oral cavity, sometimes it is necessary to observe an oral state first so as to determine whether there are foreign objects in teeth and a size of the foreign objects and then perform a corresponding rinsing. However, the existing water flosser can only clean the teeth. The existing water flosser cannot automatically recognize conditions in the oral cavity and be automatically controlled and cannot control an intensity of rinsing. In this way, on the one hand, if a force of a water jet is too small, some foreign objects will not be removed or will not be removed completely, and a cleaning effect will not be obvious. On the other hand, if the force is too strong or excessively frequent rinsing occurs, the mouth or face will be injured, making the user feel uncomfortable, which will reduce customer satisfaction.

### BRIEF SUMMARY OF THE DISCLOSURE

The present disclosure provides a controlling method of a water flosser, a water flosser control system, and a water flosser to solve the deficiencies in the background, which can automatically rinse based on a teeth area and ensure a rinsing effect and enhances comfort of a user.

In order to solve the technical problem, technical solutions of the present disclosure are as follows.

A controlling method of a water flosser comprises: obtaining an oral image through an image acquiring device installed on the water flosser, and controlling a water outlet nozzle of the water flosser to discharge water based on the oral image acquired by the image acquiring device.

In a preferred embodiment, the controlling the water outlet nozzle of the water flosser to discharge the water based on the oral image acquired by the image acquiring device comprises: recognizing a teeth area based on the oral image; when the teeth area is recognized, controlling the water outlet nozzle of the water flosser to discharge the water; and when the teeth area is not recognized, controlling the water outlet nozzle of the water flosser to not discharge the water.

In a preferred embodiment, the controlling the water outlet nozzle of the water flosser to discharge the water based on the oral image acquired by the image acquiring device comprises: recognizing a teeth area based on the oral image; when the teeth area is recognized and an area of foreign objects in the teeth area is equal to or greater than a judgment threshold, controlling the water outlet nozzle of the water flosser to discharge the water; and when at least one of the teeth area is not recognized or the area of foreign objects in the teeth area is not equal to or greater than the judgment threshold, controlling the water outlet nozzle of the water flosser to not discharge the water.

In a preferred embodiment, the controlling the water outlet nozzle of the water flosser to discharge the water based on the oral image acquired by the image acquiring device comprises: recognizing a teeth area based on the oral image; when the teeth area is recognized, further detecting whether the water outlet nozzle of the water flosser faces the teeth area; when the water outlet nozzle of the water flosser faces the teeth area, controlling the water outlet nozzle of the water flosser to discharge the water; and when the water outlet nozzle of the water flosser does not face the teeth area, controlling the water outlet nozzle of the water flosser to not discharge the water or controlling the water outlet nozzle of the water flosser to rotate to face the teeth area and then to discharge the water.

In a preferred embodiment, the controlling the water outlet nozzle of the water flosser to discharge the water based on the oral image acquired by the image acquiring device comprises one of a first method or a second method. The first method comprises: recognizing a teeth area based on the oral image; when the teeth area is recognized and an area of foreign objects in the teeth area is greater than or equal to a judgment threshold, further detecting whether the water outlet nozzle of the water flosser faces the teeth area; when the water outlet nozzle of the water flosser faces the teeth area, controlling the water outlet nozzle of the water flosser to discharge the water; and when the water outlet nozzle of the water flosser does not face the teeth area, controlling the water outlet nozzle of the water flosser to not discharge the water or controlling the water outlet nozzle of the water flosser to rotate to face the teeth area and then to discharge the water. The second method comprises: recognizing the teeth area based on the oral image; when the teeth area is recognized and the water outlet nozzle of the water flosser faces the teeth area, further detecting whether the area of the foreign objects in the teeth area is greater than or equal to the judgment threshold; when the area of the foreign objects in the teeth area is greater than or equal to the judgment threshold, controlling the water outlet nozzle of the water flosser to discharge the water; and when the area of the foreign objects in the teeth area is not greater than or equal to the judgment threshold, controlling the water outlet nozzle of the water flosser to not discharge water.

In a preferred embodiment, the controlling the water outlet nozzle of the water flosser to discharge the water based on the oral image acquired by the image acquiring device comprises: controlling a water outlet flow rate of the water outlet nozzle of the water flosser based on the oral image acquired by the image acquiring device to increase, decrease, or remain unchanged.

In a preferred embodiment, the controlling the water outlet flow rate of the water outlet nozzle of the water flosser based on the oral image acquired by the image acquiring device comprises: recognizing a teeth area based on the oral image acquired by the image acquiring device; and when the teeth area is recognized and the teeth area contains foreign objects, controlling the water outlet flow rate of the water outlet nozzle of the water flosser to increase, decrease, or remain unchanged according to an area of the foreign objects.

In a preferred embodiment, the controlling the water outlet nozzle of the water flosser to discharge the water based on the oral image acquired by the image acquiring device comprises: controlling a water outlet force of the water outlet nozzle of the water flosser based on the oral image acquired by the image acquiring device to increase, decrease, or remain unchanged.

In a preferred embodiment, the controlling the water outlet force of the water outlet nozzle of the water flosser based on the oral image acquired by the image acquiring device comprises: recognizing a teeth area based on the oral image acquired by the image acquiring device; and when the teeth area is recognized and the teeth area contains foreign objects, controlling the water outlet force of the water outlet nozzle of the water flosser to increase, decrease, or remain unchanged according to an area of the foreign objects.

In a preferred embodiment, the recognizing the teeth area based on the oral image comprises: performing Fourier transform on the oral image f0 obtained by the image acquiring device to obtain a transformed image F0; performing image enhancement on the transformed image F0 to obtain a teeth edge image F0′ including high-frequency components; processing the teeth edge image F0′ through a first band-pass filter to obtain a filtered image F0″; performing inverse Fourier transform on the filtered image F0″ to obtain a transformed image f0′; and traversing the transformed image f0′ to obtain an image f1 marked with the teeth area.

In a preferred embodiment, the recognizing the area of the foreign objects based on the teeth area comprises: mapping image f1 marked with the teeth area to the oral image f0 obtained by the image acquiring device; intercepting a teeth portion of the oral image f0 to obtain an image f2; performing Fourier transform on the image f2 to obtain an image F2; processing the image F2 through a second high-pass filter to obtain a filtered image F2′; performing inverse Fourier transform on the filtered image F2′ to obtain an image f2′; performing binarization processing on the image f2′, marking the foreign objects as black dots, and obtaining an image f2″; and traversing the image f2″, and counting the area of the foreign objects.

In a preferred embodiment, an area covered by a jet from the water outlet nozzle at least partially overlaps with an imaging area of the image acquiring device.

In a preferred embodiment, the image acquiring device is a visible light imaging device, an infrared thermal imaging device, or an ultraviolet imaging device, and the image acquiring device is detachably connected to or non-detachably connected to the water flosser.

A water flosser control system comprises an oral image acquiring module configured to acquire an oral image through an image acquiring device installed on a water flosser; and a control module configured to control a water outlet nozzle of the water flosser to discharge water based on the oral image acquired by the image acquiring device.

In a preferred embodiment, the control module is configured to recognize a teeth area based on the oral image acquired by the image acquiring device, when the teeth area is recognized, the control module controls the water outlet nozzle of the water flosser to discharge the water, and when the teeth area is not recognized, the control module controls the water outlet nozzle of the water flosser to not discharge the water.

In a preferred embodiment, the control module is configured to recognize a teeth area based on the oral image acquired by the image acquiring device, when the teeth area is recognized and an area of foreign objects in the teeth area is greater than or equal to a judgment threshold, the control module controls the water outlet nozzle of the water flosser to discharge the water, and when at least one of the teeth area is not recognized and the area of foreign objects in the teeth area is not greater than or equal to the judgment threshold the control module controls the water outlet nozzle of the water flosser to not discharge the water.

In a preferred embodiment, the control module is configured to recognize a teeth area based on the oral image acquired by the image acquiring device, and when the teeth area is recognized, the control module is further configured to detect whether the water outlet nozzle of the water flosser faces the teeth area, when the water outlet nozzle of the water flosser faces the teeth area, the control module controls the water outlet nozzle of the water flosser to discharge the water, and when the water outlet nozzle of the water flosser does not face the teeth area, the control module controls the water outlet nozzle of the water flosser to not discharge the water or the control module controls the water outlet nozzle of the water flosser to rotate to face the teeth area and then to discharge the water.

In a preferred embodiment, the control module is configured to recognize a teeth area based on the oral image acquired by the image acquiring device, when the teeth area is recognized and an area of foreign objects in the teeth area is greater than or equal to a judgment threshold, the control module is further configured to detect whether the water outlet nozzle of the water flosser faces the teeth area, when the water outlet nozzle of the water flosser faces the teeth area, the control module controls the water outlet nozzle of the water flosser to discharge the water, and when the water outlet nozzle of the water flosser does not face the teeth area, the control module controls the water outlet nozzle of the water flosser to not discharge the water or the control module controls the water outlet nozzle of the water flosser to rotate to face the teeth area to discharge the water; or when the teeth area is recognized and the water outlet nozzle of the water flosser faces the teeth area, the control module is further configured to detect whether the area of the foreign objects in the teeth area is greater than or equal to the judgment threshold, when the area of the foreign objects in the teeth area is greater than or equal to the judgment threshold, the control module controls the water outlet nozzle of the water flosser to discharge the water, and when the area of the foreign objects in the teeth area is not greater than or equal to the judgment threshold, the control module controls the water outlet nozzle of the water flosser to not discharge water.

In a preferred embodiment, the control module is configured to control a water outlet flow rate of the water outlet nozzle of the water flosser based on the oral image acquired by the image acquiring device to increase, decrease, or remain unchanged.

In a preferred embodiment, the control module is configured to recognize a teeth area based on the oral image acquired by the image acquiring device, and when the teeth area is recognized and the teeth area contains foreign objects, the control module controls the water outlet flow rate of the water outlet nozzle of the water flosser according to an area of the foreign objects.

In a preferred embodiment, the control module is configured to control a water outlet force of the water outlet nozzle of the water flosser based on the oral image acquired by the image acquiring device to increase, decrease, or remain unchanged.

In a preferred embodiment, the control module is configured to recognize a teeth area based on the oral image acquired by the image acquiring device, and when the teeth area is recognized and the teeth area contains foreign objects, the control module controls the water outlet force of the water outlet nozzle of the water flosser according to an area of the foreign objects.

In a preferred embodiment, an area covered by a jet from the water outlet nozzle at least partially overlaps with an imaging area of the image acquiring device.

In a preferred embodiment, the image acquiring device is a visible light imaging device, an infrared thermal imaging device, or an ultraviolet imaging device, and the image acquiring device is detachably connected to or non-detachably connected to the water flosser.

A water flosser comprises a body, the water outlet nozzle, the image acquiring device, and the water flosser control system. The water outlet nozzle and the image acquiring device are installed on the body.

A water flosser comprises a body, the water outlet nozzle, the image acquiring device, a memory, a processor, and a computer program stored in the memory and configured to run on the processor. The water outlet nozzle and the image acquiring device are installed on the body. When the processor executes the computer program, the controlling method of the water flosser.

Compared with the existing techniques, the technical solution has the following advantages.

(1) The present disclosure obtains the oral image of an oral cavity through the image acquiring device installed on the water flosser and controls the water outlet nozzle of the water flosser to discharge or to not discharge water based on the oral image of the oral cavity, so as to realize automatic control of rinsing according to the oral image of the oral cavity, which works without manual control and adjustment, which is convenient for users;

(2) The present disclosure can quickly recognize the teeth area and a corresponding area of the foreign objects from the oral image, and when the water outlet nozzle of the water flosser faces the teeth area, the water outlet nozzle of the water flosser is controlled to perform rinsing based on the area of the foreign objects to ensure that the jet from the water outlet nozzle faces the teeth area. An intensity of rinsing can be automatically adjusted according to the area of the foreign objects, while fully cleaning the teeth, preventing damage to the mouth or face, and improving user comfort and satisfaction;

(3) The image acquiring device of the present disclosure is preferably a visible light imaging device, an infrared thermal imaging device, or an ultraviolet imaging device, and the water outlet port of the water outlet nozzle is located in the imaging area of the image acquiring device. A rinsing position of the water flosser corresponds to an imaging positon of the image acquiring device to ensure that when the teeth area appears in a center of the oral image of the image acquiring device, the water outlet nozzle faces the teeth area at this time, and there is no need to adjust a facing direction of the water outlet nozzle.

The above description is only an overview of the technical solution of the present disclosure, in order to be able to understand the technical means of the present disclosure more clearly, so that it can be implemented according to the content of the description, and in order to make the above and other purposes, features, and advantages of the present disclosure more obvious and easy It is understood that specific embodiments of the present disclosure are listed below.

The above and other objects, advantages, and features of the present disclosure will be more apparent to those skilled in the art from the following detailed description of specific embodiments of the present disclosure in conjunction with the accompanying drawings.

### BRIEF DESCRIPTION OF THE DRAWINGS

FIG. 1 illustrates a flow chart of a controlling method of a water flosser according to an embodiment of the present disclosure.

FIG. 2 illustrates a schematic diagram of a water outlet nozzle facing a teeth area according to an embodiment of the present disclosure.

FIG. 3 illustrates a flow chart of recognizing the teeth region according to an embodiment of the present disclosure.

FIG. 4 illustrates a flow chart of acquiring an area of foreign objects according to an embodiment of the present disclosure.

FIG. 5 illustrates a structural block diagram of a water flosser control system of an embodiment of the present disclosure.

FIG. 6 illustrates a side view of the water flosser of an embodiment of the present disclosure.

FIG. 7 illustrates a perspective view of the water flosser of an embodiment of the present disclosure.

FIG. 8 illustrates a cross-sectional view of a water outlet nozzle separately installed on a body of an embodiment of the present disclosure.

FIG. 9 illustrates a cross-sectional view of an endoscope separately installed on the body of an embodiment of the present disclosure.

FIG. 10 illustrates a cross-sectional view of a water tank in a retracted position according to an embodiment of the present disclosure.

FIG. 11 illustrates a cross-sectional view of a water tank in an extended position according to an embodiment of the present disclosure.

FIG. 12 illustrates a control principle diagram of the water flosser according to an embodiment of the present disclosure.

### DETAILED DESCRIPTION OF THE EMBODIMENTS

The present disclosure will be further described below in combination with the accompanying drawings and embodiments.

The technical solutions in the embodiments of the present disclosure will be clearly and completely described below with reference to the accompanying drawings in the embodiments of the present disclosure; obviously, the described embodiments are only a part of the embodiments of the present disclosure, not all of the embodiments. The embodiments of the present disclosure, and all other embodiments obtained by those of ordinary skill in the art without creative work, fall within the protection scope of the present disclosure.

In the description of the present disclosure, it should be noted that the terms “comprising”, “comprises”, or any other variation thereof are intended to encompass non-exclusive inclusion, such that a process, method, article, or device comprising a series of elements not only including those elements, but also other elements not expressly listed or inherent to such a process, method, article, or apparatus. Without further limitation, an element qualified by the phrase “comprising a . . . ” does not preclude the presence of additional identical elements in a process, method, article, or apparatus that includes the element.

In the description of the present disclosure, it should be noted that the orientations or positional relationships indicated by the terms “upper”, “lower”, “inner”, “outer”, “top/bottom”, etc. are based on the orientations shown in the accompanying drawings. The positional relationship is only for the convenience of describing the present disclosure and simplifying the description, rather than indicating or implying that the referred device or element must have a specific orientation, be constructed and operated in a specific orientation, and therefore should not be construed as a limitation of the present disclosure. Furthermore, the terms “first” and “second” are used for descriptive purposes only and should not be construed to indicate or imply relative importance.

In the description of the present disclosure, it should be noted that, unless otherwise expressly specified and limited, the terms “installation”, “provided with”, “sleeve/connection”, “connection”, etc., should be understood in a broad sense, such as “connection”, which can be a fixed connection, a detachable connection, or an integral connection, a mechanical connection, an electrical connection, a direct connection, an indirect connection through an intermediate medium, or an internal connection between two components. For those of ordinary skill in the art, the specific meanings of the above terms in the present disclosure can be understood in specific situations.

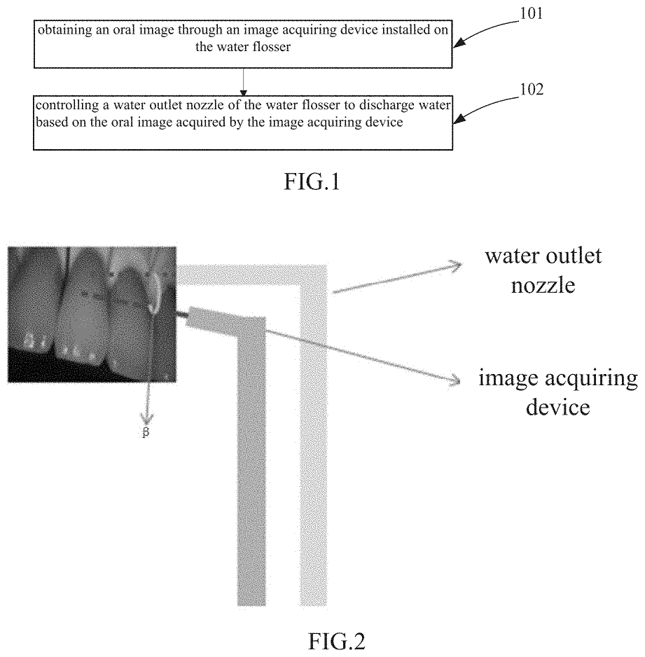

Referring to FIGS. 1-4, a controlling method of a water flosser of the present disclosure comprises:

101: obtaining an oral image through an image acquiring device installed on the water flosser; and

102: controlling a water outlet nozzle of the water flosser to discharge water based on the oral image acquired by the image acquiring device.

Through the controlling method, the water outlet nozzle of the water flosser can be automatically controlled to work based on the oral image, without manual control and adjustment, which is convenient for users. In particular, for elderly users and child users, using manual control increases difficulty of use and leads to some potential use risks. With the controlling method of the present disclosure, teeth can be automatically cleaned by placing the water flosser into an oral cavity, which reduces the difficulty of use and increases safety.

Further, foreign objects in this embodiment comprise tartar and/or food residues.

In one embodiment, the controlling the water outlet nozzle of the water flosser to discharge water based on the oral image acquired by the image acquiring device comprises:

recognizing a teeth area based on the oral image; when the teeth area is recognized, the water outlet nozzle of the water flosser is controlled to discharge water; otherwise, the water outlet nozzle of the water flosser is controlled not to discharge water.

In this embodiment, only when the teeth area is recognized based on the oral image, is the water outlet nozzle of the water flosser controlled to discharge water to rinse the teeth, so as to prevent the water outlet nozzle from also discharging water for rinsing when a non-teeth area is recognized based on the oral image. If the water is discharged without recognizing the teeth area, the water will be wasted, and it will cause certain damage to the oral cavity, such as causing the user to feel pain or even causing oral injury.

In one embodiment, the controlling the water outlet nozzle of the water flosser to discharge water based on the oral image acquired by the image acquiring device comprises:

recognizing a teeth area based on the oral image; when the teeth area is recognized and an area of the foreign objects is equal to or greater than a judgment threshold, the water outlet nozzle of the water flosser is controlled to discharge water; otherwise, the water outlet nozzle of the water flosser is controlled not to discharge water.

In this embodiment, only when the teeth area is recognized based on the oral image and the area of the foreign objects in the teeth area is greater than or equal to the judgment threshold, is the water outlet nozzle of the water flosser controlled to discharge water to rinse the teeth. It can be seen that, in order to control the water outlet nozzle of the water flosser to discharge water, the teeth area needs to be recognized, and it is also necessary to judge a size of the area of the foreign objects in the teeth area. Only when the area of the foreign objects reaches the judgment threshold, is the water outlet nozzle of the water flosser controlled to discharge water. This embodiment can prevent rinsing too frequently.

It should be noted that the judgment threshold may be a preset value or a customized value, which is specifically set according to actual use, which is not limited in this embodiment.

In one embodiment, the controlling the water outlet nozzle of the water flosser to discharge water based on the oral image acquired by the image acquiring device comprises:

recognizing a teeth area based on the oral image; when the teeth area is recognized, further detecting whether the water outlet nozzle of the water flosser faces the teeth area; when the water outlet nozzle of the water flosser faces the teeth area, controlling the water outlet nozzle of the water flosser to discharge water; otherwise, controlling the water outlet nozzle of the water flosser not to discharge water, or controlling the water outlet nozzle of the water flosser to rotate to face the teeth area and then to discharge water.

In this embodiment, only when the teeth area is recognized based on the oral image and the water outlet nozzle of the water flosser faces the teeth area, is the water outlet nozzle of the water flosser controlled to discharge water to rinse the teeth. It can be seen that, in order to control the water outlet nozzle of the water flosser to discharge water, the teeth area needs to be recognized, and it is also necessary to determine whether the water outlet nozzle of the water flosser faces the teeth area. Only when the water outlet nozzle of the water flosser faces the teeth area, is the water outlet nozzle of the water flosser controlled to discharge water. This embodiment further ensures that the teeth are rinsed.

In one embodiment, the controlling the water outlet nozzle of the water flosser to discharge water based on the oral image acquired by the image acquiring device comprises:

recognizing a teeth area based on the oral image; when the teeth area is recognized and an area of the foreign objects in the teeth area is greater than or equal to a judgment threshold, further detecting whether the water outlet nozzle of the water flosser faces the teeth area; when the water outlet nozzle of the water flosser faces the teeth area, controlling the water outlet nozzle of the water flosser to discharge water; otherwise, controlling the water outlet nozzle of the water flosser not to discharge water, or controlling the water outlet nozzle of the water flosser to rotate to face the teeth area and then to discharge water.

In this embodiment, only when the teeth area is recognized based on the oral image, the area of the foreign objects area in the teeth area is greater than or equal to the judgment threshold, and the water outlet nozzle of the water flosser faces the teeth area, is the water flosser controlled to discharge water to rinse the teeth. It can be seen that, in order to control the water outlet nozzle of the water flosser to discharge water, the teeth area needs to be recognized, and it is also necessary to determine whether the area of foreign objects in the teeth area is greater than or equal to the judgment threshold and whether the water outlet nozzle of the water flosser faces the teeth area. Only when the three conditions are met, is the water outlet nozzle of the water flosser controlled to discharge water to rinse the teeth. This embodiment not only ensures accuracy of rinsing, but also prevents excessive rinsing.

Of course, the three conditions being met at the same time can also be:

recognizing the teeth area based on the oral image; when the teeth area is recognized and the water outlet nozzle of the water flosser faces the teeth area, further detecting whether the area of the foreign objects in the teeth area is greater than or equal to the judgment threshold; when the area of the foreign objects in the teeth area is greater than or equal to the judgment threshold, controlling the water outlet nozzle of the water flosser to discharge water; otherwise, controlling the water outlet nozzle of the water flosser not to discharge water.

Either of the above two methods can achieve the above purpose of the disclosure, and the specific method can be selected according to needs.

For the water nozzle of the water flosser that cannot be rotated or cannot be rotated automatically, when it is determined that the water outlet nozzle of the water flosser does not face the teeth area, the water outlet nozzle of the water flosser is controlled to not perform rinsing, thereby preventing accidental rinsing. But at this time, a sound prompt or a light prompt can be given to prompt that the foreign objects are detected but the water outlet nozzle does not face the teeth area.

For the water outlet nozzle that can be rotated automatically, when the foreign objects are detected but the water outlet nozzle does not face the teeth area, an offset angle or an offset displacement between the water outlet nozzle and the teeth area can be calculated first, then the water outlet nozzle can be driven by a driving mechanism to rotate to face the teeth area, and finally the water outlet nozzle of the water flosser is controlled to perform rinsing based on the area of the foreign objects.

Further, the water outlet nozzle may be driven by a motor to rotate or perform rinsing.

In this embodiment, a method of determining whether the water outlet nozzle of the water flosser faces the teeth area is as follows.

Referring to FIG. 2, when the teeth area appears in a center of the oral image acquired by the image acquiring device, a facing direction of the water outlet nozzle faces the teeth area at this time. Therefore, through a structure in which an angle 13 between the water outlet nozzle and a lens of the image acquiring device and an imaging distance d are provided (i.e., known), it can be ensured that when the teeth area appears in the center of the oral image, the water outlet nozzle also faces the teeth. Therefore, determining whether the water outlet nozzle faces the teeth area becomes determining whether a center area of the oral image is the teeth area.

Specifically, a center point of the oral image is taken as a reference origin, and all vectors of the tooth contours relative to the origin are added. When an absolute length of a resulting vector is less than a preset threshold, it is determined that the teeth are not in the center of the oral image, otherwise it is determined that the teeth are in the center of the oral image, that is, the water outlet nozzle faces the teeth.

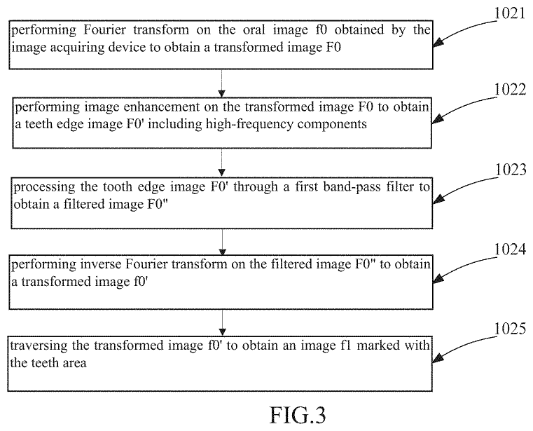

Referring to FIG. 3, the recognizing the teeth area based on the oral image specifically comprises:

1021: performing Fourier transform on the oral image f0 obtained by the image acquiring device to obtain a transformed image F0;

In this step, the oral image f0 obtained by the image acquiring device is transformed from time domain to frequency domain;

1022: performing image enhancement on the transformed image F0 to obtain a teeth edge image F0′ including high-frequency components;

1023: processing the tooth edge image F0′ through a first band-pass filter to obtain a filtered image F0″;

In this step, the first band-pass filter can continuously perform a learning mechanism to accurately process the tooth edge.

Specifically, according to the actual oral image, it can be known that the teeth area should be within a certain frequency range after Fourier transformation (1021). Assuming that the range is (wl, wh), the following learning mechanism is established.

1000 oral images are acquired in advance, and each oral image is processed as follows:

Step 1: numbering each oral image as n in sequence, and n ranges from 1 to 1000;

Step 2 manually marking the teeth area on each oral image;

Step 3: performing Fourier transform on each oral image to find a frequency domain of a marked area of each oral image;

Step 4: recording a label n and a corresponding frequency range (wn1, wn2);

Step 5: repeatedly performing Step 3 and Step 4, a total of 1000 times, to get 1000 groups of frequency ranges, (w11, w22), (w21, w22) . . . . . .

Step 6: removing abnormal data from the 1000 groups of data (i.e., the 1000 groups of frequency ranges) in Step 5, and ranking the 1000 groups of data according to the labels to output a sequence Wi; the sequence Wi comprises several groups of frequency ranges that are not removed;

Step 7: taking an average of an upper frequency limit and a lower frequency limit of the sequence Wi, respectively, obtaining a mean value range (wl, wh) by calculation, and obtaining a mean square error 61 of wl and a root mean square di of wh by calculation;

Step 8: taking the sequence Wi, the mean value range (wl, wh), and d and di together as output data of the learning mechanism, that is, parameters of the first band-pass filter.

Further, when the obtained oral images continuously increase, the parameters of the first band-pass filter are constantly corrected through the above-mentioned method.

1024: performing inverse Fourier transform on the filtered image F0″ to obtain a transformed image f0′.

In this step, the processed image (i.e., the transformed image f0′) is transformed back to the time domain.

1025: traversing the transformed image f0′ to obtain an image f1 marked with the teeth area.

After being processed by the first band-pass filter, only the corresponding frequency part of the image (i.e., the oral image) is retained, that is, only teeth outline information is retained on a rendering image. Considering that the band-passed image is not visually continuous, the following processing should be done:

Step 1: traversing all pixels and removing outliers;

Step 2: setting a window parameter wnd, and eroding the image to make it a connected contour area;

Step 3: traversing each connected domain, removing sub-areas located other connected domains, obtaining a teeth contour map, and obtaining the teeth area.

It should be noted that the above is only a specific implementation manner of recognizing the teeth area, and other existing teeth area recognition methods can also be applied in the present disclosure, which is not specifically limited in the embodiment of the present disclosure.

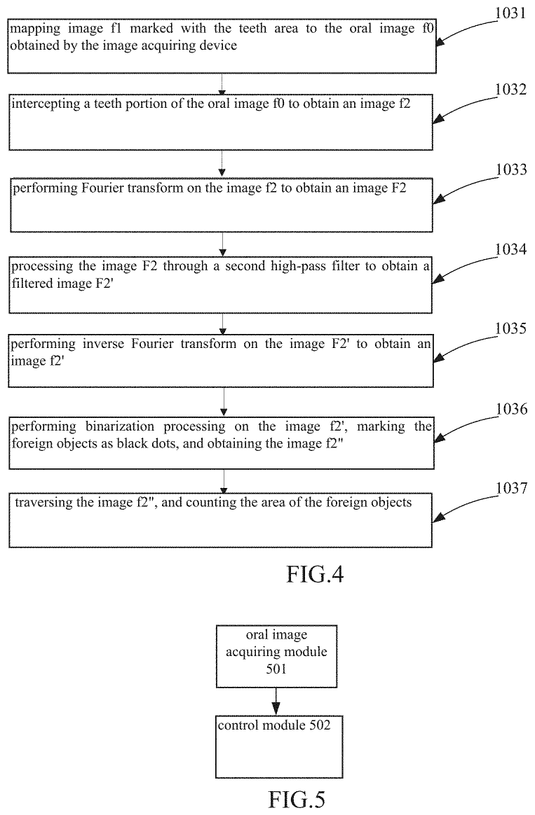

Referring to FIG. 4, obtaining the area of the foreign objects based on the teeth area that is recognized specifically comprises:

1031: mapping image f1 marked with the teeth area to the oral image f0 obtained by the image acquiring device.

1032: intercepting a teeth portion of the oral image f0 to obtain an image f2.

Specifically, comprising:

Step 1: creating a frame of image, the size of the frame of image is the same as an original image (i.e., oral image f0);

Step 2: by scanning an outline image of the teeth area in 1025, copying pixels with the same coordinates in the original image to the frame of image;

Step 3: the frame of image being used as an original image of the teeth portion, which is the image f2.

1033: performing Fourier transform on the image f2 to obtain an image F2.

It can be seen from the above description that, in this step, only the teeth portion is subjected to Fourier transform to make it be transferred from time domain into frequency domain.

1034: processing the image F2 through a second high-pass filter to obtain a filtered image F2′.

In this step, the second high-pass filter can continuously perform a learning mechanism.

Specifically, 1000 oral images were acquired in advance. According to the actual oral images, the area of foreign objects in the teeth should be within a certain frequency range after Fourier transform. Assuming that the certain frequency range is (wb, +cc), the following learning mechanism is established.

Each image is processed as follows:

Step 1: numbering each oral image as n in sequence, and n ranges from 1 to 1000;

Step 2 manually marking a portion of the foreign objects, and counting an area of the portion of the foreign objects S1, S2, S3 . . . ;

Step 3: performing Fourier transform on each image to find a frequency domain of the portion of the foreign objects which is marked on each oral image;

Step 4: recording the label n and the corresponding frequency range (wn, +GO;

Step 5: repeatedly performing Step 3 and Step 4, a total of 1000 times, to get 1000 groups of frequency ranges, (w1, +∞), (w2, +∞)

Step 6: removing abnormal data from the 1000 groups of data (i.e., the 1000 groups of frequency ranges) in Step 5, sorting by the labels, and outputting a sequence Wi; the sequence Wi comprises several groups of frequency ranges that are not removed;

Step 7: taking an average of an upper limit of frequency in the sequence Wi, obtaining a mean value range (wb, +co) by calculation, and obtaining a mean square error 6b of wb by calculation;

Step 8: removing abnormal points from the area of the portion of the foreign objects, and taking an average Sth1 and a mean square error σthl, where Sth1 is the judgment threshold.

Step 9: taking the sequence Wi, the frequency range (i.e., the mean value range) (wb, +co) and 6b together as output data of the learning mechanism, that is, parameters of the second high-pass filter.

Further, when the obtained oral images continuously increase, the parameters are constantly corrected through the above processing flow to improve the accuracy.

1035: performing inverse Fourier transform on the filtered image F2′ to obtain an image f2′.

In this step, the enhanced image is converted back to time domain.

1036: performing binarization processing on the image f2′, marking the foreign objects as black dots, and obtaining the image f2″.

Specifically, in order to facilitate calculation of the portion of the foreign objects, it is necessary to perform binary image processing, as follows:

Step 1: removing outliers of the image f2′ obtained after inverse Fourier transform;

Step 2: traversing the image, and converting all pixels into gray scale values;

Step 3: setting a threshold, and setting pixels on both sides of the threshold to black and white;

Step 4: outputting a new image processed by pixel points, on which the portion of the foreign objects is marked, and obtaining the image f2″.

1037: traversing the image f2″, and counting the area of the foreign objects.

It should be noted that the above is only a specific implementation manner of acquiring the area of foreign objects, and other existing methods for acquiring the area of foreign objects can also be applied in the present disclosure, which are not specifically limited in the embodiments of the present disclosure.

Further, this embodiment can control a change of a water outlet flow rate of the water outlet nozzle of the water flosser based on the oral image acquired by the image acquiring device so as to enable the water outlet flow rate to increase, decrease, or remain unchanged; and/or, this embodiment can control a change of a water outlet force of the water outlet nozzle of the water flosser based on the oral image acquired by the image acquiring device to enable the water outlet force to be larger, smaller, or unchanged.

Specifically, judging conditions for controlling the water outlet flow rate and/or the water outlet force may be the area of the foreign objects, a type of the foreign objects, a thickness of the foreign objects, and the like.

If the judging conditions is the area of the foreign objects, the specific method may be to control the water outlet flow rate and/or the water outlet force of the water outlet nozzle of the water flosser based on the oral image acquired by the image acquiring device, comprising:

recognizing the teeth area based on the oral image acquired by the image acquiring device; when the teeth area is recognized and the teeth area contains the foreign objects, controlling the water outlet flow rate and/or the water outlet force of the water outlet nozzle of the water flosser according to the area of the foreign objects.

The controlling the water outlet nozzle of the water flosser to discharge water for rinsing based on the area of the foreign objects, comprises:

based on the area of the foreign objects, controlling the water outlet nozzle of the water flosser to perform rinsing in different gear positions.

Specifically, when the area of the foreign objects S is greater than or equal to the judgment threshold Sth1, the water outlet nozzle of the water flosser is controlled to perform rinsing.

In this embodiment, according to the area of the foreign objects S, the water flosser can be divided into three rinsing gear positions, as follows.

When Sth1<S<S1, the area of the foreign objects is small, and a low gear position is started (e.g., a motor speed is low, rinsing for 1 second);

When S1<S<Sh, the area of the foreign objects is medium, and a middle gear position is started (the motor speed is medium, rinsing for 3 seconds);

When S>Sh, the area of the foreign objects is large, and a high gear position is started (the motor speed is high, rinsing for 5 seconds).

The above is only a schematic embodiment, and the specific gear positions and the rinsing time can be set according to actual applications, which are not limited in the embodiment of the present disclosure.

Further, an area covered by a jet from the water outlet nozzle at least partially overlaps with an imaging area of the image acquiring device. Preferably, a water outlet port of the water outlet nozzle is located in the imaging area of the image acquiring device.

Specifically, by specially designing a relative position of the water flosser and the image acquiring device, an imaging position of the image acquiring device corresponds to a rinsing position of the water flosser, so that there is no need to adjust the relative position. This design can also be used to view a rinsing effect of the water flosser in real time through the image acquiring device, which is very clear and intuitive.

In this embodiment, the image acquiring device can be a visible light imaging device, an infrared thermal imaging device, or an ultraviolet imaging device. The image acquiring device can be detachably connected to or non-detachably connected to the water flosser.

The visible light imaging device can be an endoscope. The endoscope is a detection instrument that integrates traditional optics, ergonomics, precision machinery, modern electronics, mathematics, and software. The endoscope has image sensors, optical lenses, light source lighting devices, and mechanical devices. The endoscope is installed on the water flosser as the image acquiring device, which can not only acquire images of the oral cavity, but also facilitate the real-time observation of the oral conditions and the observation of the rinsing effect, which is very clear and intuitive.

It can be understood that other image acquiring devices can also be used in the present disclosure, as long as images of the oral cavity can be acquired and the above-mentioned functions can be realized.

A water flosser control system of the present disclosure, referring to FIG. 5, comprises:

an oral image acquiring module 501 configured to acquire the oral image through the image acquiring device installed on the water flosser; and

a control module 502 configured to control the water outlet nozzle of the water flosser to discharge water based on the oral image acquired by the image acquiring device.

In one embodiment, the control module 502 is specifically configured to recognize the teeth area based on the oral image acquired by the image acquiring device, and when the teeth area is recognized, the control module 502 controls the water outlet nozzle of the water flosser to discharge water; otherwise, the control module 502 controls the water outlet nozzle of the water flosser to not discharge water.

In one embodiment, the control module 502 is specifically configured to recognize the teeth area based on the oral image acquired by the image acquiring device, and when the teeth area is recognized and the area of the foreign objects in the teeth area is greater than or equal to the judgment threshold, the control module 502 controls the water outlet nozzle of the water flosser to discharge water; otherwise, the control module 502 controls the water outlet nozzle of the water flosser to not discharge water.

In one embodiment, the control module 502 is specifically configured to recognize the teeth area based on the oral image acquired by the image acquiring device. When the teeth area is recognized, it is further detected whether the water outlet nozzle of the water flosser faces the teeth area, if so, the water outlet nozzle of the water flosser is controlled to discharge water; otherwise, the water outlet nozzle of the water flosser is controlled not to discharge water, or the water outlet nozzle of the water flosser is controlled to be rotated to face the teeth area and then to discharge water.

In one embodiment, the control module 502 is specifically configured to recognize teeth area based on the oral image acquired by the image acquiring device. When the teeth area is recognized and the area of the foreign objects in the teeth area is greater than or equal to the judgment threshold, it is further detected whether the water outlet nozzle of the water flosser faces the teeth area, if so, the water outlet nozzle of the water flosser is controlled to discharge water; otherwise, the water outlet nozzle of the water flosser is controlled to not discharge water, or the water outlet nozzle of the water flosser is controlled to be rotated to face the teeth area to discharge water.

Alternatively, the control module 502 is specifically configured to:

recognize the teeth area based on the oral image acquired by the image acquiring device. When the teeth area is recognized and the water outlet nozzle of the water flosser faces the teeth area, it is further determined whether the area of the foreign objects in the teeth area is greater than or equal to the judgment threshold, if so, the water outlet nozzle of the water flosser is controlled to discharge water; otherwise, the water outlet nozzle of the water flosser is controlled to not discharge water.

The control module 502 is specifically configured to:

control the water outlet flow rate of the water outlet nozzle of the water flosser based on the oral image acquired by the image acquiring device to increase, decrease, or remain unchanged.

The controlling the water outlet flow rate of the water outlet nozzle of the water flosser based on the oral image acquired by the image acquiring device comprises:

recognizing the teeth area based on the oral image acquired by the image acquiring device; when the teeth area is recognized and the teeth area contains the foreign objects, controlling the water outlet flow rate of the water outlet nozzle of the water flosser according to the area of the foreign objects.

The control module 502 is also specifically configured to:

based on the oral image acquired by the image acquiring device, control the water outlet force of the water outlet nozzle of the water flosser to increase, decrease, or remain unchanged.

The controlling the water outlet force of the water outlet nozzle of the water flosser based on the oral image acquired by the image acquiring device comprises:

recognizing the teeth area based on the oral image acquired by the image acquiring device; when the teeth area is recognized and the teeth area contains the foreign objects, controlling the water outlet force of the water outlet nozzle of the water flosser according to the area of the foreign objects.

The area covered by the jet from the water outlet nozzle at least partially overlaps with the imaging area of the image acquiring device.

The image acquiring device is a visible light imaging device, an infrared thermal imaging device, or an ultraviolet imaging device. The image acquiring device is detachably connected to or non-detachably connected to the water flosser.

For the specific implementation of the oral image acquiring module 501 and the control module 502, reference may be made to a controlling method of a water flosser, which will not be repeated here.

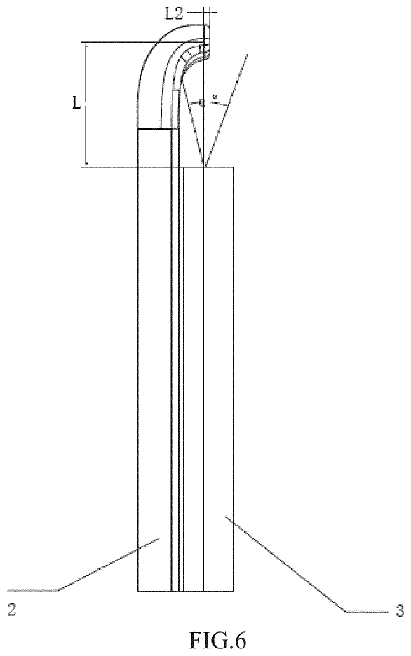

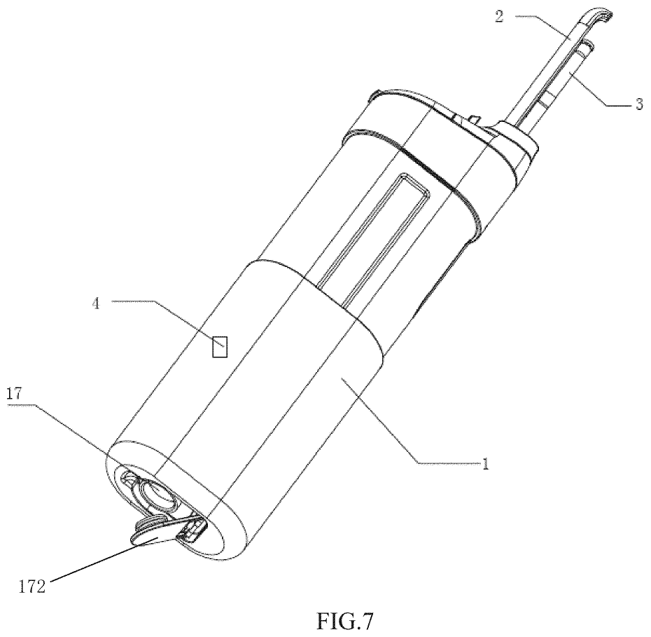

An embodiment of a water flosser of the present disclosure, referring to FIGS. 6-13, comprises a body 1, a water outlet nozzle 2, and an image acquiring device 3. The water outlet nozzle 2 and the image acquiring device 3 are installed on the body 1. The water flosser further comprises the water flosser control system as abovementioned. In this embodiment, the water flosser control system is implemented by a controller 4 (e.g., microcontroller unit (MCU)). The image acquiring device 3 may be a visible light imaging device, an infrared thermal imaging device, or an ultraviolet imaging device.

A focal length range of the image acquiring device 3 may be a to b.

According to different image acquiring devices 3, values of a and b are different. A lens surface of the image acquiring device 3 is lower than a water outlet port of the water outlet nozzle 2. The lens surface of the image acquiring device 3 faces the water outlet nozzle 2, and the water outlet port of the water outlet nozzle 2 is located in an imaging area of the image acquiring device 3.

After the above design, the imaging area of the image acquiring device 3 corresponds to an area to be rinsed by the water outlet nozzle 2, and the area to be rinsed by the water outlet nozzle 2 always appears in the imaging area of the image acquiring device 3. Preferably, the water outlet nozzle 2 and the area to be rinsed by the water outlet nozzle 2 always appear in the imaging area of the image acquiring device 3. In this way, when the area of the foreign objects is detected and the water outlet nozzle 2 of the water flosser faces the teeth area, the controller 4 controls the water outlet nozzle 2 of the water flosser to perform rinsing through a motor 14.

Specifically, a height difference L between the lens surface of the image acquiring device 3 and the water outlet port of the water outlet nozzle 2 satisfies: a<L<b. A vertical distance L2 between the water outlet port of the water outlet nozzle 2 and a central axis of the image acquiring device 3 satisfies: 0≤L2≤tan(α°/2)*L; wherein: α° represents a dispersion angle of the endoscope (i.e., the image acquiring device 3).

Further, in order to facilitate a user storing the water outlet nozzle 2 and the image acquiring device 3, the water outlet nozzle 2 and the image acquiring device 3 are respectively detachably connected to the body 1. The water outlet nozzle 2 is directly connected to the body 1 in an insertion manner, and a bottom of the image acquiring device 3 comprises a connector that forms a mechanical connection and an electrical connection to the body 1, so that the image acquiring device 3 can take electricity from the body 1 and be fixedly connected to the body 1. Such a connector may be in a mode of magnetic attraction and conductive elastic piece 31, which belongs to the prior art and will not be repeated here. As a simple replacement of this embodiment, the image acquiring device 3 can also be fixedly connected to the body 1 and cannot be removed.

In this embodiment, in order to facilitate the user storing the water outlet nozzle 2 and the image acquiring device 3 which have been removed, the body 1 has a chamber for accommodating the image acquiring device 3 and the water outlet nozzle 2. In this way, the user does not need to prepare an additional storage box, and it is very convenient to carry when traveling.

In addition, the water flosser is a portable water flosser, so the body 1 has a water tank 17, and a bottom of the body 1 has a cover 172 for opening or closing the water tank 17. In this way, all the required components can be integrated on the body 1, and a storage space for the water flosser is small, which is further convenient for the user to carry.

In addition, the body 1 comprises a lighting switch 12 and a photographing switch 13.

When in use, the user presses the lighting switch 12 to control the image acquiring device 3 to be activated or deactivated; after the image acquiring device 3 is activated, the photographing switch 13 is pressed to obtain the imaging area of the image acquiring device 3.

The lighting switch 12 and the photographing switch 13 may be buttons or touch switches.

In order to allow the user to intuitively know a current rinsing gear position, the body 1 further comprises one or more indicator lights for indicating the current rinsing gear position. A number of indicator lights is the same as that of the rinsing gear positions, and when the current rinsing gear position is increased or decreased, the number of indicator lights will increase or decrease accordingly.

In order to facilitate the user observing his/her own oral cavity, an application (APP) installed on a smart terminal is provided, which establishes a wireless connection between the APP and the image acquiring device 3 and displays the imaging area of the image acquiring device 3 on the APP in real time (or, displays an oral photo taken by the image acquiring device 3 on the APP). In this way, the user can observe his/her own oral cavity in real time when rinsing his/her teeth, or take images of his/her oral cavity and keep the images for careful observation.

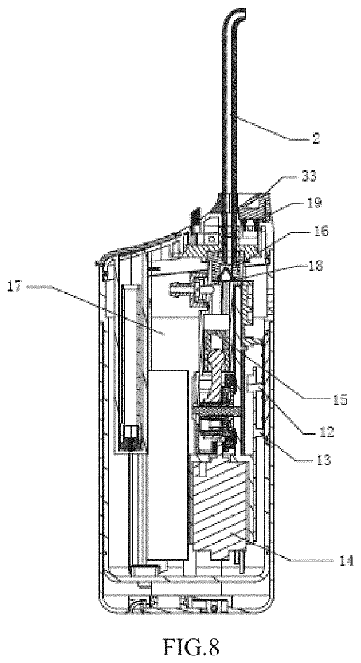

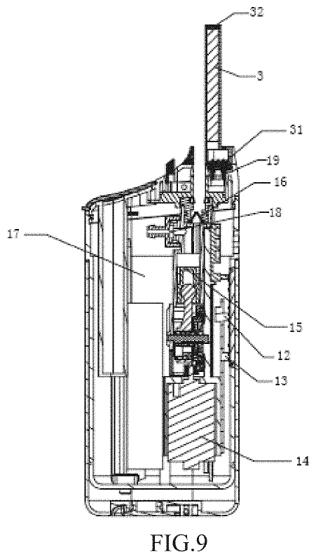

Further referring to FIGS. 8 and 9, in order to complete a water outlet function, the motor 14 is disposed inside the body 1 to drive a piston assembly 15, and the motor 14 drives the piston assembly 15 to reciprocate to pump water in the water tank 17 into a pipeline 151 inside the body 1. The pipeline 151 is in communication with the water outlet nozzle 2 through a duckbill-shaped valve 16, so that a backflow of the water in the water outlet nozzle 2 can be avoided. The water tank 17 is in communication with the pipeline 151 through a check valve 18, which also prevents the water in the pipeline 151 from flowing back into the water tank 17.

In order to complete an imaging function of the image acquiring device 3, a circuit board of the image acquiring device 3 is disposed inside of the body 1, and a conductive contact 19 is disposed on the body 1, which is electrically connected to a conductive elastic piece 31 of the image acquiring device 3. The conductive contact 19 is connected to the circuit board by wires. An end of the image acquiring device 3 facing the oral cavity comprises a waterproof lens 32 for protecting a lens of the image acquiring device 3. Meanwhile, when the image acquiring device 3 is not installed on the body 1, a waterproof cover 33 is installed on the body 1 to cover the conductive contact 19.

The lighting switch 12 and the photographing switch 13 are connected to the circuit board of the image acquiring device 3, and the circuit board of the image acquiring device 3 is connected to the controller 4 to send the acquired oral image to the controller 4.

In order to complete a rinsing function, a circuit board of the water flosser is disposed inside of the body 1, and the circuit board of the water flosser is connected to the controller 4. The controller 4 controls the water outlet nozzle 2 to perform rinsing or stop rinsing through the circuit board of the water flosser.

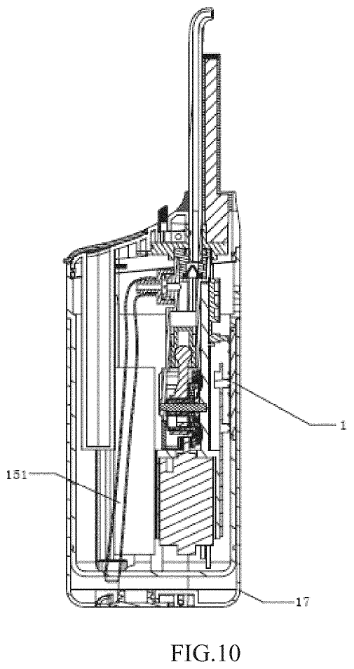

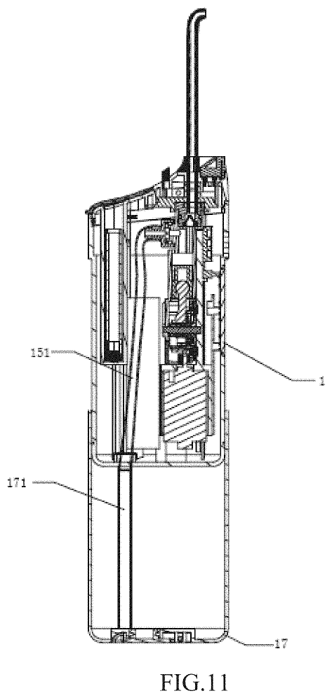

Further referring to FIGS. 10 and 11, in this embodiment, an outer wall of the body 1 is further slidably connected to an inner wall of the water tank 17, so that the water tank 17 moves between an extended position and a retracted position. In order to realize that the water in the water tank 17 can be pumped by the piston assembly 15 in the body 1, a telescopic tube 171 is arranged in the water tank 17. An upper end of the telescopic tube 171 is kept in communication with the pipeline 151 in the body 1, and a lower end of the telescopic tube 171 extends or contracts correspondingly with movement of the water tank 17. Since the water tank 17 is slidably connected to the body 1, the water tank 17 can be slid to the retracted position to reduce a storage space of the water flosser during storage, and an effective volume of the water tank 17 can be increased by sliding the water tank 17 to the extended position during use.

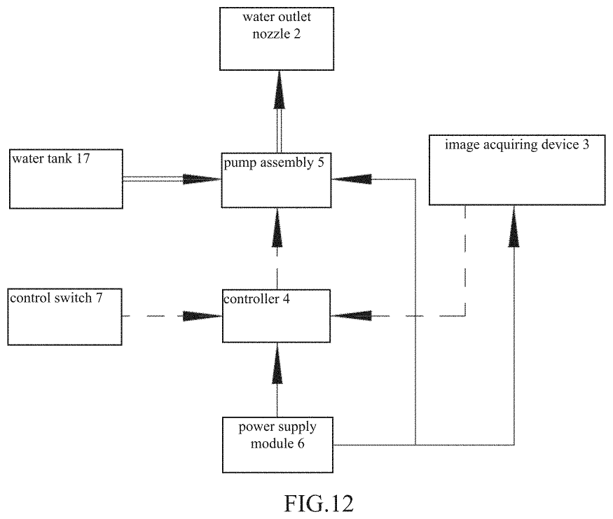

Referring to FIG. 12, a control principle of the water flosser of the present disclosure is as follows: a power supply module 6 is connected to the controller 4, the image acquiring device 3, and a pump assembly 5 to separately supply power to the controller 4, the image acquiring device 3, and the pump assembly 5. A control switch 7 connected to the controller 4 is configured to control the controller 4 to start working. The image acquiring device 3 is connected to the controller 4 to send the acquired oral image to the controller 4. The controller 4 is configured to recognize the oral image, and when the controller 4 recognizes the oral image that meets some specific conditions, the controller 4 controls the pump assembly 5, and the pump assembly 5 controls the water in the water tank 17 to be sprayed from the water outlet nozzle 2 to rinse the teeth.

It can be understood that although the above description describes automatic control of the controller as an example, “controlling the water outlet nozzle of the water flosser to discharge water or not to discharge water based on the oral image acquired by the image acquiring device” can actually be controlled by manual control. Alternatively, functions of the automatic control and the manual control can simultaneously exist. When the automatic control and the manual control coexist, if a control switch 7 is turned on, the automatic control is preferentially used, and if the control switch 7 is turned off, the manual control is used. When using the manual control, a manual control switch can be set on the body of the water flosser, and a prompt device can be set at the same time. When it is recognized that the teeth need to be washed or the water flow rate needs to be adjusted according to the oral image, the prompt device can be used to prompt the user, and the user controls or adjusts according to the prompts.

In an embodiment of the water flosser of the present disclosure, the water flosser further comprises a memory, a processor, and a computer program stored in the memory and configured to run on the processor, and the processor implements steps of the controlling method of the water flosser when the processor executes the computer program.

Specifically, the processor is configured to:

obtain the oral images through the image acquiring device installed on the water flosser; and

control the water outlet nozzle of the water flosser based on the oral image acquired by the image acquiring device.

In this embodiment, the memory and the processor may be integrated on the controller and implemented by the controller.

In one embodiment, the water flosser further comprises the APP installed on the smart terminal, and the APP establishes a wireless connection to the image acquiring device 3 to display the imaging area of the image acquiring device 3 on the APP in real time, or to display the oral photo taken by the image acquiring device 3 on the APP.

In one embodiment, a connection position of the image acquiring device 3 and the body 1 is located on a side of the water outlet nozzle 2 facing the jet, and the lens surface of the image acquiring device 3 is lower than a water outlet surface of the water outlet nozzle 2.

In one embodiment, a connection position of the image acquiring device 3 and the body 1 is located on a side of the water outlet nozzle 2 facing away from the jet, and the lens surface of the image acquiring device 3 is higher than a water outlet surface of the water outlet nozzle 2.

The aforementioned embodiments are merely some embodiments of the present disclosure, and the scope of the disclosure is not limited thereto. Thus, it is intended that the present disclosure cover any modifications and variations of the presently presented embodiments provided they are made without departing from the appended claims and the specification of the present disclosure.

What is claimed is:

1. A controlling method of a water flosser, comprising:
obtaining an oral image through an image acquiring device installed on the water flosser, and
controlling a water outlet nozzle of the water flosser to discharge water based on the oral image acquired by the image acquiring device.

2. The controlling method of the water flosser according to claim 1, wherein:
the controlling the water outlet nozzle of the water flosser to discharge the water based on the oral image acquired by the image acquiring device comprises:
recognizing a teeth area based on the oral image;
when the teeth area is recognized, controlling the water outlet nozzle of the water flosser to discharge the water; and
when the teeth area is not recognized, controlling the water outlet nozzle of the water flosser to not discharge the water.

3. The controlling method of the water flosser according to claim 1, wherein:
the controlling the water outlet nozzle of the water flosser to discharge the water based on the oral image acquired by the image acquiring device comprises:
recognizing a teeth area based on the oral image;
when the teeth area is recognized and an area of foreign objects in the teeth area is equal to or greater than a judgment threshold, controlling the water outlet nozzle of the water flosser to discharge the water; and
when at least one of the teeth area is not recognized or the area of foreign objects in the teeth area is not equal to or greater than the judgment threshold, controlling the water outlet nozzle of the water flosser to not discharge the water.

4. The controlling method of the water flosser according to claim 1, wherein:
the controlling the water outlet nozzle of the water flosser to discharge the water based on the oral image acquired by the image acquiring device comprises:
recognizing a teeth area based on the oral image;
when the teeth area is recognized, further detecting whether the water outlet nozzle of the water flosser faces the teeth area;
when the water outlet nozzle of the water flosser faces the teeth area, controlling the water outlet nozzle of the water flosser to discharge the water; and
when the water outlet nozzle of the water flosser does not face the teeth area, controlling the water outlet nozzle of the water flosser to not discharge the water or controlling the water outlet nozzle of the water flosser to rotate to face the teeth area and then to discharge the water.

5. The controlling method of the water flosser according to claim 1, wherein:
the controlling the water outlet nozzle of the water flosser to discharge the water based on the oral image acquired by the image acquiring device comprises one of a first method or a second method:
the first method comprises:
recognizing a teeth area based on the oral image;
when the teeth area is recognized and an area of foreign objects in the teeth area is greater than or equal to a judgment threshold, further detecting whether the water outlet nozzle of the water flosser faces the teeth area;
when the water outlet nozzle of the water flosser faces the teeth area, controlling the water outlet nozzle of the water flosser to discharge the water; and
when the water outlet nozzle of the water flosser does not face the teeth area, controlling the water outlet nozzle of the water flosser to not discharge the water or controlling the water outlet nozzle of the water flosser to rotate to face the teeth area and then to discharge the water, or
the second method comprises:
recognizing the teeth area based on the oral image;
when the teeth area is recognized and the water outlet nozzle of the water flosser faces the teeth area, further detecting whether the area of the foreign objects in the teeth area is greater than or equal to the judgment threshold;
when the area of the foreign objects in the teeth area is greater than or equal to the judgment threshold, controlling the water outlet nozzle of the water flosser to discharge the water; and
when the area of the foreign objects in the teeth area is not greater than or equal to the judgment threshold, controlling the water outlet nozzle of the water flosser to not discharge water.

6. The controlling method of the water flosser according to claim 1, wherein:
the controlling the water outlet nozzle of the water flosser to discharge the water based on the oral image acquired by the image acquiring device comprises:
controlling a water outlet flow rate of the water outlet nozzle of the water flosser based on the oral image acquired by the image acquiring device to increase, decrease, or remain unchanged.

7. The controlling method of the water flosser according to claim 6, wherein:
the controlling the water outlet flow rate of the water outlet nozzle of the water flosser based on the oral image acquired by the image acquiring device comprises:
recognizing a teeth area based on the oral image acquired by the image acquiring device; and
when the teeth area is recognized and the teeth area contains foreign objects, controlling the water outlet flow rate of the water outlet nozzle of the water flosser to increase, decrease, or remain unchanged according to an area of the foreign objects.

8. The controlling method of the water flosser according to claim 1, wherein:
the controlling the water outlet nozzle of the water flosser to discharge the water based on the oral image acquired by the image acquiring device comprises:
controlling a water outlet force of the water outlet nozzle of the water flosser based on the oral image acquired by the image acquiring device to increase, decrease, or remain unchanged.

9. The controlling method of the water flosser according to claim 8, wherein:
the controlling the water outlet force of the water outlet nozzle of the water flosser based on the oral image acquired by the image acquiring device comprises:
recognizing a teeth area based on the oral image acquired by the image acquiring device; and
when the teeth area is recognized and the teeth area contains foreign objects, controlling the water outlet force of the water outlet nozzle of the water flosser to increase, decrease, or remain unchanged according to an area of the foreign objects.

10. The controlling method of the water flosser according to claim 2, wherein:
the recognizing the teeth area based on the oral image comprises:
performing Fourier transform on the oral image f0 obtained by the image acquiring device to obtain a transformed image F0;
performing image enhancement on the transformed image F0 to obtain a teeth edge image F0′ including high-frequency components;
processing the teeth edge image F0′ through a first band-pass filter to obtain a filtered image F0″;
performing inverse Fourier transform on the filtered image F0″ to obtain a transformed image f0′; and
traversing the transformed image f0′ to obtain an image f1 marked with the teeth area.

11. The controlling method of the water flosser according to claim 3, wherein:
the recognizing the area of the foreign objects based on the teeth area comprises:
mapping image f1 marked with the teeth area to the oral image f0 obtained by the image acquiring device;
intercepting a teeth portion of the oral image f0 to obtain an image f2;
performing Fourier transform on the image f2 to obtain an image F2;
processing the image F2 through a second high-pass filter to obtain a filtered image F2′;
performing inverse Fourier transform on the filtered image F2′ to obtain an image f2′;
performing binarization processing on the image f2′, marking the foreign objects as black dots, and obtaining an image f2″; and
traversing the image f2″, and counting the area of the foreign objects.

12. The controlling method of the water flosser according to claim 1, wherein:
an area covered by a jet from the water outlet nozzle at least partially overlaps with an imaging area of the image acquiring device.

13. The controlling method of the water flosser according to claim 1, wherein:
the image acquiring device is a visible light imaging device, an infrared thermal imaging device, or an ultraviolet imaging device, and
the image acquiring device is detachably connected to or non-detachably connected to the water flosser.

14. A water flosser control system, comprising:
an oral image acquiring module configured to acquire an oral image through an image acquiring device installed on a water flosser; and
a control module configured to control a water outlet nozzle of the water flosser to discharge water based on the oral image acquired by the image acquiring device.

15. The water flosser control system according to claim 14, wherein:
the control module is configured to recognize a teeth area based on the oral image acquired by the image acquiring device,
when the teeth area is recognized, the control module controls the water outlet nozzle of the water flosser to discharge the water, and
when the teeth area is not recognized, the control module controls the water outlet nozzle of the water flosser to not discharge the water.

16. The water flosser control system according to claim 14, wherein:
the control module is configured to recognize a teeth area based on the oral image acquired by the image acquiring device,
when the teeth area is recognized and an area of foreign objects in the teeth area is greater than or equal to a judgment threshold, the control module controls the water outlet nozzle of the water flosser to discharge the water, and
when at least one of the teeth area is not recognized and the area of foreign objects in the teeth area is not greater than or equal to the judgment threshold the control module controls the water outlet nozzle of the water flosser to not discharge the water.

17. A water flosser, comprising:
a body,
the water outlet nozzle,
the image acquiring device, and
the water flosser control system according to claim 14, wherein:
the water outlet nozzle and the image acquiring device are installed on the body, and
an area covered by a jet from the water outlet nozzle at least partially overlaps with an imaging area of the image acquiring device.

18. A water flosser, comprising:
a body,
the water outlet nozzle,
the image acquiring device,
a memory,
a processor, and
a computer program stored in the memory and configured to run on the processor, wherein:
the water outlet nozzle and the image acquiring device are installed on the body,
an area covered by a jet from the water outlet nozzle at least partially overlaps with an imaging area of the image acquiring device, and
when the processor executes the computer program, the controlling method of the water flosser according to claim 1 is performed.

19. The water flosser according to claim 17, wherein:
a connection position of the image acquiring device and the body is located on a side of the water outlet nozzle facing the jet, and
a lens surface of the image acquiring device is lower than a water outlet surface of the water outlet nozzle.

20. The water flosser according to claim 17, wherein:
a connection position of the image acquiring device and the body is located on a side of the water outlet nozzle facing away from the jet, and
a lens surface of the image acquiring device is higher than a water outlet surface of the water outlet nozzle.
